(19) United States
(12) Patent Application Publication
(10) Pub. No.: US 2026/0207974 A1
(43) Pub. Date: Jul. 23, 2026

(54) Ultrasonic Surface Optimizer

(71) Applicant: Enrique Rangel Gonzalez, Coyoacan (MX)

(72) Inventor: Enrique Rangel Gonzalez, Coyoacan (MX)

(21) Appl. No.: 19/567,091

(22) Filed: Mar. 14, 2026

Related U.S. Application Data
(63) Continuation-in-part of application No. 18/219,900, filed on Jul. 10, 2023, now abandoned.

(30) Foreign Application Priority Data
Dec. 15, 2022 (MX) MX/U/2022/000597

(51) Int. Cl. A61N 7/00

(52) U.S. Cl. CPC A61N 7/00; A61N 2007/0008

(57) ABSTRACT

The present invention refers to an ultrasonic surface optimizer or ultrasonic probe to which a rough texture has been added to the entire tip of the sonotrode. The device allows fragmenting or emulsifying of subcutaneous soft tissues optimally. The probe includes an ultrasonic vibrational energy source, a longitudinal handle with a proximal end and a distal end, adjacent thereto along the same axis a longitudinal stem (sonotrode) with a proximal end and a distal end. Also, there is a connection at the proximal end of the longitudinal handle for connecting the ultrasonic probe to the ultrasonic vibratory energy source. Finally, the probe includes a point at the distal end of the longitudinal stem with one or more grooves near the tip, wherein the grooves are transverse to the longitudinal axis, and the distal end of the sonotrode includes a textured or rough finish.

### CROSS-REFERENCES TO RELATED APPLICATIONS

This application is a continuation-in-part of U.S. patent application Ser. No. 18/219,900, filed on Jul. 10, 2023 which claims benefit of Mexican Patent Application No. MX/U/2022/000597, filed on Dec. 15, 2022, the disclosures of which are hereby incorporated by reference in their entirety to provide continuity of disclosure to the extent such disclosures are not inconsistent with the disclosure herein.

### DESCRIPTION

### Technical Field of the Invention

The present invention belongs to the field of invasive medical devices that are intended for short-term use, specifically for class II and class III classification, according to the level of risk associated with their use. From this classification, it is considered an active medical device whose operation depends on a source of electrical energy and acts as a conversion of said energy.

The present invention also refers to surgically invasive medical devices such as an ultrasonic probe as it penetrates into the body through an incision in the body surface, wherein the ultrasonic probe comes into contact with the adipose tissue and adjacent connective tissue, and wherein the ultrasonic energy emitted by the ultrasonic probe acts upon these tissues, emulsifying the adipose tissue and thereby promoting tissue retraction-all through a surgical procedure.

### Background of the Invention

The present invention relates to the area of surgical instruments, particularly surgical-type medical devices that come into contact with soft tissue, such as an ultrasonic surface optimizer or an ultrasonic probe.

The surgical intervention known as liposuction or liposculpture is based on the extraction of excess adipose tissue, a procedure that requires a cannula or ultrasonic probe that is introduced through incisions in the skin to tear or crush the adipose tissue, which is then removed thanks to a suction method.

The cannulas currently used for the adipose tissue detachment procedure are characterized by being small-diameter metal tubes, with closed and rounded tips; some embodiments may also feature fenestrations or holes near the distal end. They are inserted into the patient through incisions in the skin and then the surgeon performs continuous movements of the cannula, which induces the removal of adipose tissue, as well as the disruption of other tissues, which causes irregularities, bruising, bleeding, swelling and numbness as side effects of said procedure.

Ultrasound-assisted liposuction (UAL) technique is used particularly in the liposuction procedure and works as follows:
a) High-frequency ultrasonic sound waves are used to obtain a homogenized mixture. These sound waves are transmitted through a solid stem-type tube called a sonotrode (since it transmits ultrasonic vibrations longitudinally) and which allows the emulsification process of adipose tissue to be carried out through the cavitation effect.
b) The sonotrode is introduced through small incisions made in a subdermal plane to break up the tissues and detach the fat cells.
c) On the other hand, the power source assists a transducer that converts the energy and transmits it to the sonotrode thereby causing the sonotrode to have mechanical vibrations at the same frequency.

In the state of the art there are several patents related to the invention of instruments intended to improve the liposuction or lipoplasty, ultrasound-assisted (UAL) for the removal of adipose tissue, which are described below:
U.S. Pat. No. 5,255,669 refers to an apparatus consisting of a probe that has a system for generating ultrasonic vibrations and a sheath to cover at least one outer peripheral surface of the vibrational transmission member of the ultrasonic probe, plus a fluid supply unit adjacent to a coupled portion of the sheath for supplying a fluid within a defined passage between the vibrational transmission member and the sheath. Since it doesn't mention a sonotrode with a rough texture, it can be inferred that it is a different invention from the one proposed by the ultrasonic probe of the present invention.
U.S. Patent Publication No. 2018/0021599 which refers to an apparatus for removing fat by separating lipocytes, which uses ultrasonic waves for this purpose. May include: a fat removal body part; a handpiece including an ultrasonic wave generating unit electrically connected to the fat removal body part; a tip portion provided on the handpiece: a temperature sensor unit for detecting the temperature of the tip, the temperature sensor unit being provided in a tip part; an interlocked pump part for supplying cooling water to the tip, the interlocked pump part being disposed in the grease removal body unit and being connected to the handpiece; and a controller for receiving a signal detected by the temperature sensor unit, and operating the locked pump part at a preset temperature. Although you may consider some other devices related to the fat removal apparatus, it does not mention a sonotrode with rings with a rough texture as in the present invention.
Spanish patent number ES2209119, which discloses an ultrasonic surgical device (80, 180) that includes an adapter transducer assembly (82, 182) to vibrate at an ultrasonic frequency, in response to electrical energy. A transmission component (86, 186) adapted to receive ultrasonic vibration from the transducer array and transmit ultrasonic vibration from a first end to a second end. A braking device (130, 300) configured to apply a force to the outer surface of the drive component to damp ultrasonic vibration transmitted from the first end to the second end of the drive rod. Also provided is a method for inserting a trocar cannula into a patient's tissue. The procedure includes the steps of providing an ultrasonic trocar obturator (180), having an ultrasonic waveguide (186), including an effector (188) at the distal end thereof, positioning the trocar obturator within a cannula (159), placing the trocar plug in contact with a wall of said body cavity, and ultrasonic vibration of the end effector to create a penetration opening. The method further includes advancing the ultrasonic plug (180) into said penetration opening, sensing when penetration is complete, applying the braking process (300) to the plug to damp ultrasonic vibration of the end effector, and advancing the trocar cannula into the penetration opening, thus being different from the object of protection found in the present invention.
U.S. Pat. No. 6,368,299 which refers generally to an ultrasonic probe for enhanced fragmentation consisting of a longitudinal stem having a proximal tip, a distal tip and a shaft. The shaft joins the proximal end and the distal end. The proximal end of the longitudinal stem has a longitudinal stem connector attached to it by an ultrasonic vibratory energy source. The distal end of the longitudinal stem may have a rounded or pointed tip. The shaft has one or more grooves near the tip that circumscribe the axis of the longitudinal shank. The walls of one or more slots in the axis of the longitudinal stem provide a surface area at the distal end of the ultrasonic probe in planes generally perpendicular to the axis of the ultrasonic probe. The additional surface area increases the tissue fragmentation surface area of the distal end of the ultrasonic probe without increasing the diameter of the distal end of the ultrasonic probe. Thus, one can more quickly and completely fragment or emulsify tissues with a given diameter from the distal end of the ultrasonic probe. In addition, because surface area that gets in contact with the tissue along and around the distal end of the ultrasonic probe can be reduced, the potential for tissue burns can also be reduced. However, they have certain limitations, since the diameter of the cannula is increased, larger incisions must be made in the patient or a very pronounced low relief is made to create the rings, causing the structure of the cannula to destabilize with risk of being cut off.
U.S. Patent Publication No. 2008/0194999 discloses an ultrasonic surgical device that shares the following similarities with the present invention: it includes an ultrasonic transducer for generating ultrasonic vibration, a probe whose proximal end is connected to the ultrasonic transducer and extends from the proximal end to the distal end, and a treatment section that includes at least one recess formed on the side of the probe and treats living tissue through the ultrasonic vibration generated by the ultrasonic transducer; additionally, at the tip of the sonotrode, there is a series of concentric rings interspersed with radially arranged grooves. However, in relation to the present invention, the distal end of the shank terminates in a flat, circular shape with a central orifice in the cannula portion; conversely, in the present invention, the tip shape is rounded in the form of a half-sphere. This device also functions as a cannula since it has a tubular structure, i.e., its interior is hollow, which allows drainage or administration of fluid to or from the tissue, conversely in the present invention, the interior is completely solid, as such the device in question functions as an adipose tissue emulsification and as a tissue drainage or irrigation device. Another difference between the current ultrasonic surgical device and the present invention concerns the tip surface, which in the current ultrasonic surgical device is completely smooth, whereas in the present invention, the blunt tip, rings, and grooves collectively have a rough finish that increases the contact surface area between the tissue to be treated and the device.
U.S. Patent Publication No. 20210330342A1 discloses an ultrasonic surgical device that includes a probe whose proximal end is connected to the ultrasonic transducer and extends from the proximal end to the distal end. At the tip of the distal end, there is a roughened surface in the form of a crosshatch pattern generated by a plurality of U-shaped grooves arranged on the cutting surfaces (primarily upper and lower faces, secondarily lateral and frontal faces); the roughened surface generated as a result of these grooves; its upper portion has the shape of a truncated pyramid. However, this device lacks annular rings. Conversely, in the present invention, the grooves are arranged radially along the shank axis, circumscribing the rings. Additionally, the roughened surface is located in its upper portion resulting in a curved (blunt) finish rather than a truncated pyramid shape. Also, the sonotrode tip in the present invention has a half-sphere shape, whereas in the current ultrasonic surgical device, the tip resembles a rectangular prism. Also, the current ultrasonic surgical device does not include any rings, and the grooves serve to form the roughnesses whose tip has a truncated pyramid shape, whereas the present invention includes rings, with grooves circumscribing the rings (not forming the roughnesses), while the roughened surface on the blunt tips is formed by surface irregularities on the rings, grooves, and sonotrode tip.
U.S. Patent Publication No. 20110213279A1 refers to an ultrasonic device for adipose tissue emulsification, having a proximal portion, a neck (intermediate) portion, and a distal portion, with the latter segment acting on the tissue to be treated. The distal portion has a dome or mushroom shape with a convex side and a concave side; on its convex side, where ultrasonic waves are emitted, there is a roughened area (crests), while on its concave side the surface is smooth and transmits no type of ultrasonic wave. In one embodiment, the convex surface of the dome features one or more grooves that divide it into several concentric rings, each in a mushroom or dome shape, with a diameter increasing from distal to proximal, separated and interspersed with the grooves; on the convex surface of these rings there may be a roughened area (crests) which are absent on their concave faces as well as on the grooves. Conversely, in the present invention, the roughened areas are located on the surface of the blunt tip, the rings, and in the grooves. Additionally, the current ultrasonic device of this reference emits two types of ultrasonic waves on the tissue to be treated with different effects on the tissue: first, “cold ultrasonic waves” that act in a transverse plane, stretching and deforming adipocytes, fibers and cell membranes, and “hot ultrasonic waves” that impact the tissue in a longitudinal plane, acting on membrane phospholipids by exciting them and promoting emulsification. Furthermore, this ultrasonic device does not disclose that it can be used on tissue through a prior incision, which makes sense given the sonotrode morphology (dome or mushroom shape) because if the ultrasonic device of the reference is introduced into the body, there would be greater risk of trauma to adjacent tissues. Clearly, this ultrasonic device is designed to act on a body surface without direct contact with adipose tissue, performing indirect adipose tissue emulsification.
U.S. Published Application No. S20190380728 discloses an ultrasonic surgical device referred to as a sonotrode in the form of a longitudinal shank with a proximal end and a distal end; with a distal end in the form of a blunt tip, and proximally to the tip, one or more ring-shaped projections partially or fully circumscribing it. In one embodiment, the ring(s) partially or fully circumscribing the blunt tip are integrated into the lateral wall and tip via one or more perpendicular intersections, thereby giving the sonotrode tip a conical appearance. In embodiments with more than one ring, there is a groove separating each. However, in none of the embodiments is there mention of a series of roughened areas on the sonotrode tip, rings, or grooves to optimize the contact surface area between the device and the tissue to be treated.
U.S. Published US20230028334A1 discloses a device for ultrasound-assisted liposuction that emulsifies and then extracts adipose tissue; wherein the device irrigates, emulsifies, and suctions adipose tissue in a synchronized manner. The longitudinal shank of the device has a proximal end and distal end; the sonotrode is formed by several fins that transmit ultrasonic energy from the generator to the sonotrode tip and thence to the tissue to be treated; these fins are incorporated inside a longitudinal cannula that fully covers the sonotrode; through the cannula, the previously emulsified adipose tissue is displaced and the irrigation solution initially used to irrigate the tissue to be treated is also extracted. The tip of the longitudinal shank is blunt and may have multiple configurations: one or more central orifices in the tip, one or more lateral orifices circumscribing the tip, all for extracting the irrigation solution and emulsified tissue; the tip may also feature one or more grooves that may be parallel or transverse to the longitudinal axis, none alternated with rings. These grooves serve as peripheral emulsification areas. Conversely, this reference makes no reference to the presence of surface roughnesses on the tip or grooves. In general, it performs three procedures: irrigation, emulsification, and extraction.

Using the results obtained with the previous methods as a reference, an improvement was made to the ultrasonic probe of the present invention that seeks the emulsification of fat, reduction of thermal shock, and reduction of the risks of burns and other complications in patients undergoing this procedure.

In the present invention, the issue of increasing surfaces is addressed as an aspect to be explored to improve the procedure and consequently its versatility in time and cost, minimizing the current problems of overheating.

Therefore, the present invention refers to an ultrasonic surface optimizer (or ultrasonic probe) for surgical use that contains a generator for generating ultrasonic vibratory energy coupled to an elongated shank type ultrasonic probe provided with micro surfaces located at the distal end of the shank (ultrasonic probe), which allows the most efficient application in the output of ultrasound (UAL), reduces the thermal impact towards the tissue of the human body, reduces the risks of burns and complications in patients undergoing this procedure. Furthermore, this elongated cannula assists in the procedure to emulsify and homogenize fat by through an ultrasonic frequency (UAL), which is introduced into the human body through small incisions, where coordinated movements are made by the operator, to propagate the waves in order to break and detach the fat cells.

### Goals

The present invention aims to provide a surgical medical device that contains a generator for generating ultrasonic vibratory energy with an elongated ultrasonic probe, provided with micro surfaces located at the distal end of the ultrasonic probe, which allows the efficient application of ultrasound output (UAL), thereby reducing the thermal impact towards the tissue of the human body and reducing the risks of burns and complications in patients undergoing this procedure.

Another object of the present invention is to provide an ultrasound-assisted medical device, which optimizes the ultrasound output (UAL) by creating micro surfaces at distal end of the ultrasonic probe which are formed by different elements at different microscopic scales.

Finally, the present invention assists in the medical procedure to emulsify and homogenize the fat by means of an ultrasonic frequency (UAL) through smaller incisions and, where through the coordinated movements of the operator, it is possible to break and break down the fat cells.

### DESCRIPTION OF THE INVENTION

### Brief Description of the Figures

FIG. 1 is a side view in which the blunt tip, the ultrasound effect at the distal end of the longitudinal shank (ultrasonic probe) can be observed with the shading corresponding to the area designated for the roughened surface finish and optimization for ultrasound-assisted liposuction (UAL). Adjacent to the longitudinal stem is the longitudinal handle and connected to this is the ultrasonic vibratory energy source.

FIG. 2 is a frontal view of the ultrasonic probe, in which the emission of ultrasonic energy proportional to 360° is perceived.

FIG. 3 is a lateral view of the blunt tip, the grooves, and rings circumscribing the tip the ring-groove intersection with the rough finish on both elements, and the emission directions of the ultrasonic waves.

FIG. 4 illustrates in detail of the microscopic irregularities of the roughened area surface, which is formed by a series of peaks and valleys at different scales.

FIGS. 5A-5E illustrate the details of the orientation variations that a textured (rough) surface may exhibit, which are categorized respectively as: Parallel or perpendicular to the direction of the grooves (FIG. 5A); According to the bisector of the angles formed by the directions of the grooves (FIG. 5B); In any direction (FIG. 5C); Radial (FIG. 5D); and Normal to a radius (FIG. 5E).

FIG. 6 illustrates a comparison between an ultrasonic probe without an optimization surface (smooth) (left image) and a ultrasonic probe with an optimization surface (roughened surface) (right image).

FIG. 7 illustrates a magnified image of a smooth ultrasonic probe surface (left image) versus a magnified image of a rough ultrasonic probe surface (right image).

FIG. 8 illustrates that through the application of the roughened surface at the distal end of the ultrasonic probe, it is possible to make active areas the areas that are not commonly used, where a series of peaks and valleys of differing dimensions can be observed that show angles along the length of the ultrasonic probe wherever there is a rough surface, taking the entire face of any peak or valley, as an active surface as a striking surface or ultrasound output.

FIG. 9 illustrates the direction of the ultrasonic waves are emitted at the distal end of the ultrasonic probe, which can be observed to be emitted in all directions

FIG. 10 illustrates a smooth ultrasonic probe in operation in a diluent, where the smooth ultrasonic probe is generating few bubbles, which are dispersed, thus do not generate a large active area that optimizes ultrasound output.

FIG. 11 illustrates the ultrasonic probe having a roughened surface in operation in a diluent, where the ultrasonic probe having a roughened surface forms multiple bubbles that are emerging from the tip and descending, which generates a very large active area that optimizes the ultrasound output

### DETAILED DESCRIPTION OF THE INVENTION

The present invention refers to an ultrasonic surface optimizer (or ultrasonic probe having a roughened surface 4 for surgical use that contains a generator 20 for generating ultrasonic vibratory energy in an elongated shank-type stem 10 of ultrasonic probe 1 that assists in the procedure of emulsifying and homogenizing fat by means of ultrasonic frequency or ultrasound-assisted liposuction (UAL). The ultrasonic probe is introduced into the human body through small incisions, where coordinated movements are made by the operator, to propagate the waves, break and gradually detach the fat cells.

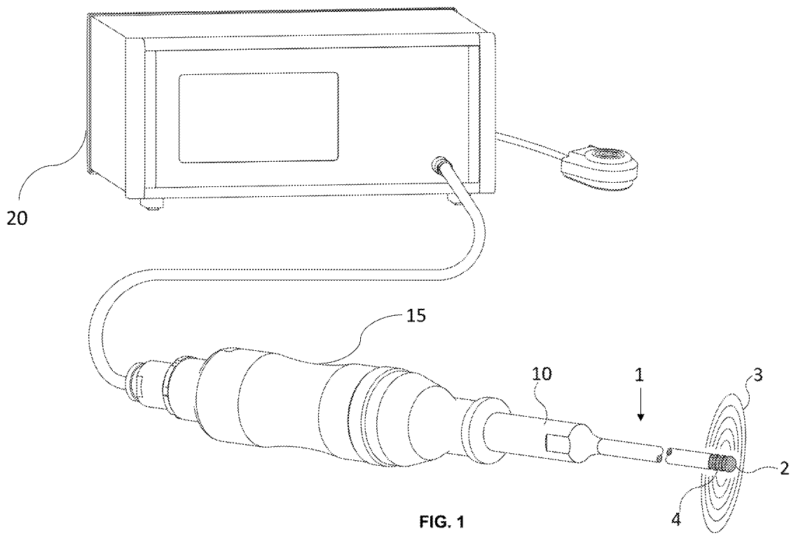

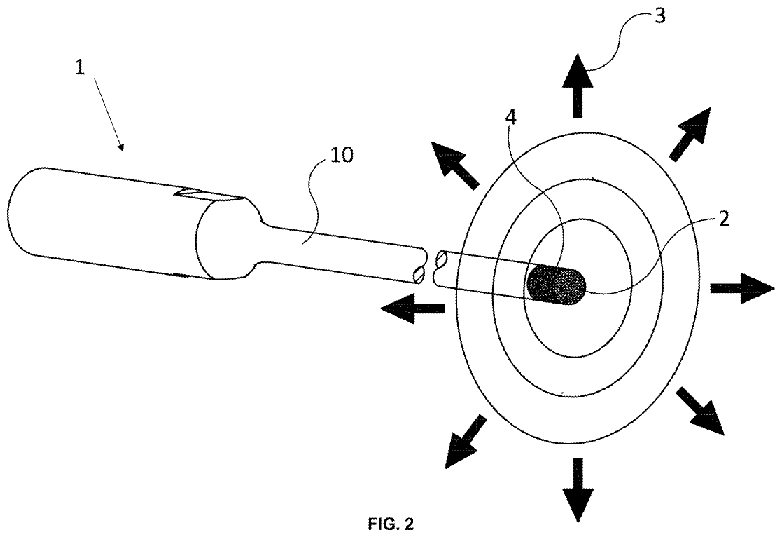

The device includes an elongated shank-type stem 10 of ultrasonic probe 1, as shown in FIGS. 1 and 2, at the end of the shank there is a plurality of grooves (3.1, 3.3, 3.5), which create a series of surfaces of different levels such as rings (3.2, 3.4), which direct energy 3 proportional to 360°, as shown in FIGS. 1 and 2. The ultrasonic probe 1 also includes a longitudinal handle 15.

More specifically, the present invention relates to an ultrasonic surface optimizer or an ultrasonic probe powered by an ultrasonic vibrational energy source 20 (FIG. 1) that assists in creating fragmentation and emulsification of subcutaneous soft tissues.

The blunt, rounded tip 2 of the ultrasonic probe 1 of the present invention features a distinctive roughened surface finish 4, designed in order to optimize the output of ultrasonic waves.

As shown in FIGS. 1, 3 and 4, the surface finish consists of repetitive deviations formed by undulations and which in turn are made up of peaks and valleys, as shown in FIG. 4, that is, it is a set of irregularities that are formed on different scales of length. On the first scale, a set of waviness deviations is present and this in turn is made up of roughness deviations made up of peaks (4.2 and 4.4) and valleys (4.1 and 4.3). This composition creates micro surfaces that generate an optimal space for the effect of mechanical hammering of ultrasound (UAL). In particular, the density of the energy is optimized along the constructed surfaces due to the increase of the surface area in planes with different orientations and in different scales, such as a corrugation height or peak (4.2), a corrugation spacing or valley (4.1), both with a certain direction, and where the roughness presents a certain height (4.4) and a certain width (4.3).

The device refers to an ultrasonic probe 1 to fragment or emulsify soft tissues, the ultrasonic probe 1 being powered by an ultrasonic vibrational energy source 20, the ultrasonic probe 1 comprises the following elements:
A. Longitudinal handle 15 with a proximal end and a distal end;
B. A longitudinal stem 10 whose axis extends from the proximal end to through the distal end;
C. An axis of the longitudinal stem 10 being aligned with the center of the longitudinal handle 15 and passing through the proximal end and the distal end of the longitudinal stem;
C. A Connection at the proximal end of the handle 15 of the ultrasonic probe 1 for connecting the longitudinal handle 15 to the source of ultrasonic vibratory energy 20; and
D. A blunt, rounded tip 2 at the distal end of the longitudinal stem 10, and one or more grooves (3.1, 3.3. and 3.5) near the tip 2.

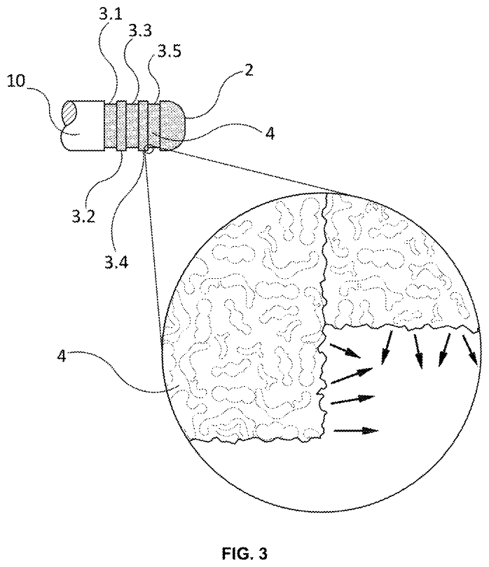

The grooves (3.2 and 3.4) are arranged generally transverse to the axis of the longitudinal stem 10, and substantially circumscribing the axis of the longitudinal stem 10, thereby increasing the tissue contact surface along and around the sides of the tip 2 and providing an additional tissue comminution surface to the tip 2 in planes generally perpendicular to the longitudinal stem 10 axis; wherein the one or more slots each have a rough bottom, each bottom, in a cross section containing the axis of the longitudinal stem 10 which is substantially parallel to the axis of the longitudinal stem 10. Each bottom of one or more grooves (3.2 and 3.4) is U-shaped in cross section (FIG. 3) and contains the axis of the longitudinal stem 10. One or more slots do completely circumscribe the shaft.

In another embodiment of the device, one or more of the grooves (3.2 and 3.4) may not completely encircle the axis of the longitudinal stem 10.

As shown in FIGS. 1 and 2, the longitudinal stem 10 is a solid structure that transmits ultrasonic waves from the proximal end to the distal end. The tip 2 located at the distal end of the longitudinal stem 10 is blunt-shaped or bullet-shaped.

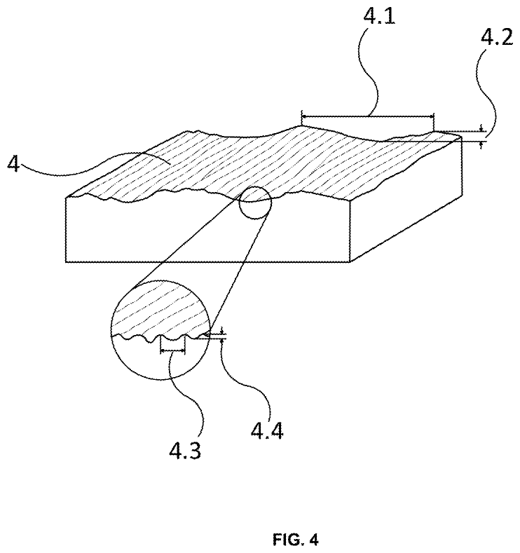

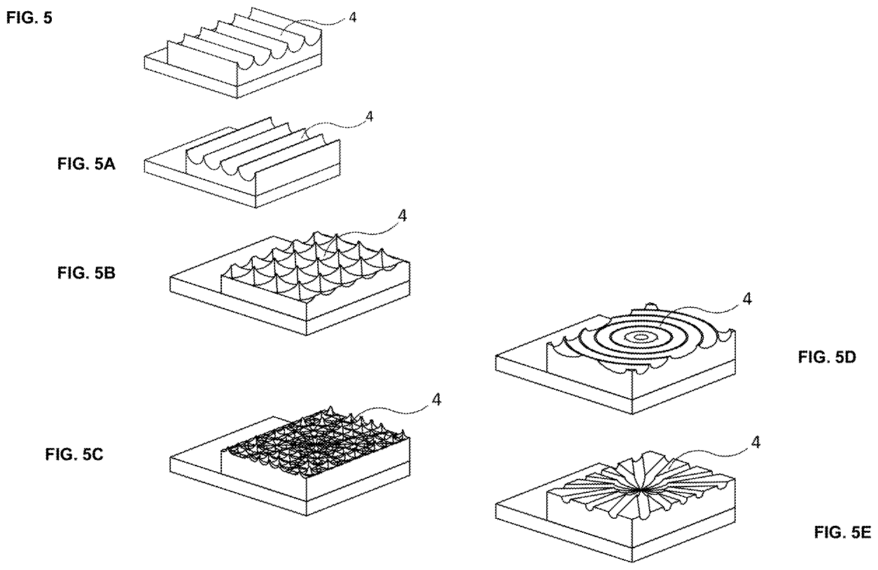

The present invention relates to the set of a roughened surface 4 (FIG. 1) on the ultrasonic probe 1 and how this feature improves the ability of the ultrasonic probe 1 to more easily fragment or emulsify the soft tissues of a patient. It is to be understood that the texture and/or orientation of the roughened surface pattern can be customized depending upon the desired emission of ultrasonic energy from the tip 2 of the ultrasonic probe 1 due to the roughened surface 4. As shown in FIG. 5, these different textures and orientations of the roughened surface 4 can be categorized into the following:
A.) Perpendicular to the direction of the grooves as illustrated in FIG. 5A.
B.) According to the bisector of the angles formed by the directions of the furrows as illustrated in FIG. 5B.
C.) In any direction as illustrated in FIG. 5C.
D.) Radial as illustrated in FIG. 5D.
E.) Normal to a radius as illustrated in FIG. 5E.

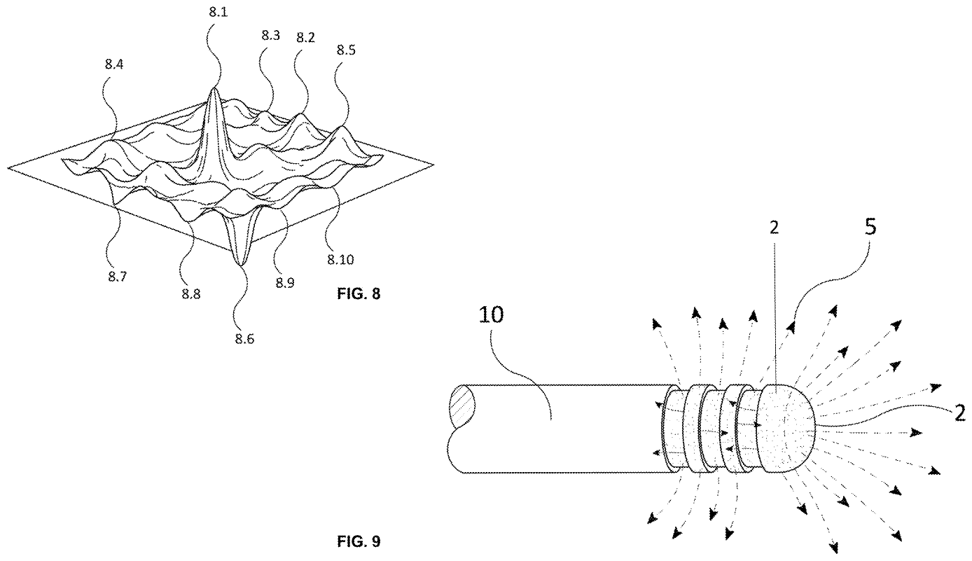

The roughened surface 4 offers an advantage over other devices with a smooth or only grooved finish and ringed ones, since through the arrangement of this micro-texture formed by peaks and valleys of the roughened surface 4, the surfaces along the tip 2 of the ultrasonic probe 1 axis are optimized, which are not used with the smooth or simply grooved, or ringed model. In addition, any transversal face that is in contact with the peaks (8.1 to 8.5) and valleys (8.6 to 8.10) (FIG. 8) that function as an extender of the longitudinal and angular surface will receive the action of the ultrasonic waves being emitted from the ultrasonic probe 1.

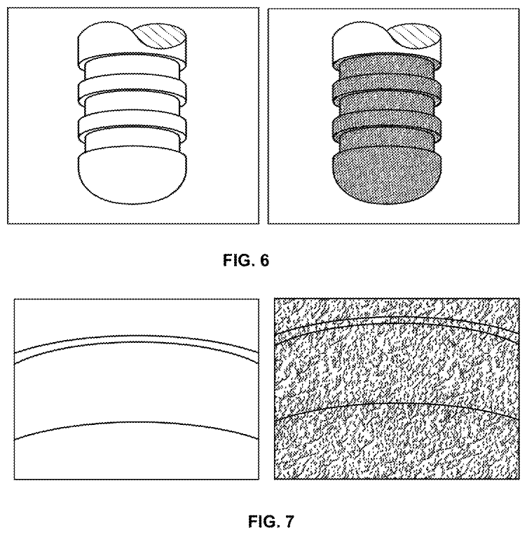

A sonotrode or ultrasonic probe “without rough surface” having a transversal wall in the rings and a longitudinal movement generates active areas, and consequently some active surfaces depending on the number of rings and grooves as illustrated in FIGS. 6, 7 and 10.

$$ACTIVE AREA = 1 / 2 SPHERE AREA + S 1 + S 2 + S 3 + S 4$$

A ultrasonic probe “with a rough surface” includes an area with micro-surfaces oriented in multiple directions that, when longitudinally moved, generate a very large active area that optimizes the ultrasound output, as illustrated in FIGS. 6, 7, 9 and 11.

In the present invention, the ultrasonic probe 1 with a tip 2 having a roughened surface 4 and constituting a set of additional surfaces has the following advantages:
A. A greater surface area is offered at different length scales, as shown in FIG. 4.
B. Increased efficiency of the ultrasound output by means of these scales, as shown in FIGS. 4 and 8, since the deviations or alterations of the undulation and roughness are found in different orientations, as shown in FIG. 5-5E.
C. Regarding the roughened surface 4 the optimization of the surface 4 as illustrated in FIGS. 3 and 8, the tapping zone 5 (FIG. 9) helps to break the adipocytes, while using less energy since the roughened surface 4 assists in creating an increased output of the ultrasound waves 3 from the ultrasonic probe 1 that are greater than any smooth surface and the ultrasound waves 3 emitted from ultrasonic probe 1 are more stable and uniform. Since less power is needed for that ultrasound wave 3 output, the ultrasonic probe 1 uses less power and generates less heat. It is to be understood that “tapping zone” 5 (FIG. 9) refers to the high-intensity areas created by the ultrasonic probe 1 where the ultrasonic waves hit a “hard” or “soft” tissue, thereby resulting in maximum cavitation.
D. The thermal impact to the tissue of the human body by the ultrasonic probe 1 can be reduced, and so, its application reduces the risks of burns and complications in patients undergoing the medical procedure.

### Comparison of Textured and Smooth Ultrasonic Probes

### Differences in Thermal Impact Reduction

Two devices called ultrasonic emulsifier, one of them HEUS brand (i.e., ultrasonic probe 1), which according to the inscriptions found on its surface was manufactured in Mexico by “Ingenieria Indemex”, SA de CV, serial number 201907087 (which features a rough surface on the sonotrode tip), and the other device brand “VASER”, which according to the inscriptions found on its surface was manufactured in the United States by “Sound Surgical Technologies LLC” with serial number: VTO4A-20100324 (whose sonotrode tip is smooth). Both devices were connected to the direct current plug located in the same room and immediately afterwards, at fourteen hours and thirty-eight minutes, the HEUS device was Heus connected to a cable attached to a handpiece or handle, which was attached to a twenty-seven centimeter long titanium ultrasonic probe 1, and then the aforementioned device was turned on by operating the power switch located on its rear part, when turning on the device, various images appeared on a screen located on its front part, including a counter or stopwatch at zeros and a power indicator at a level of 100. Then they activated the counter by pressing a pedal or external trigger and immediately began to repeatedly rub the titanium ultrasonic probe 1 roughly on a previously moistened chamois cloth and placed flat on the same table, this friction action continued until the counter marked one minute, when it was suspended and immediately afterwards they turned on the Fluke model VT04 thermal imaging camera, Ser. No. 20100324, according to the inscriptions attached to its surface, which was placed at a distance of approximately thirty centimeters aligned on the central part of the ultrasonic probe 1 that had been rubbed and after a few seconds the image of a figure indicating 43.3° C. appeared in the aforementioned chamber and then they proceeded to turn off the device.

Afterwards, a Vaser equipment was attached to a hand piece or handle, which in turn was attached to a twenty-seven centimeter long titanium sonotrode (or ultrasonic probe), and then the aforementioned device was turned on by operating the current switch located on the back, when turning on the device, in some electronic counters that are located on the front, there is a timer at zeros and another counter that indicates the power level at 100. The counter was activated by pressing a pedal or external trigger and immediately afterwards the titanium sonotrode was rubbed on a previously moistened chamois cloth and placed it flat on the same table, this friction action continued until one minute had elapsed, when it was suspended and turned on the Fluke VT04 thermographic camera again, according to the inscriptions attached to its surface. Which was placed at approximately 30 centimeters, aligned over the central part of the sonotrode that had been rubbed, and after a few seconds in the mentioned camera the image of a figure indicating 45.5° C. appeared.

### Operation Methodology

The process for the operation of the smooth surface ultrasonic probe is as follows:
A. Use a 2-liter glass flask.
B. Pour thinner into the flask.
C. Connect the smooth surface ultrasonic probe to an ultrasonic energy generation device at a power of 100.
D. Insert the smooth surface ultrasonic probe. (See left image in FIG. 6 and left image in FIG. 7).

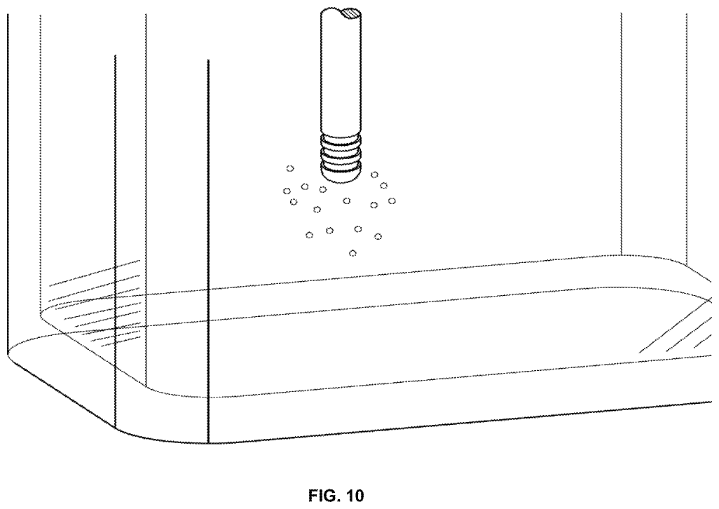

FIG. 10 shows that when the smooth surface ultrasonic probe is introduced into the thinner, a dispersed bubbling is formed, but there is no concentration of the bubbles, which causes this smooth surface ultrasonic probe to have a reduced sound emission and ultrasonic energy dispersion in relatively few directions.

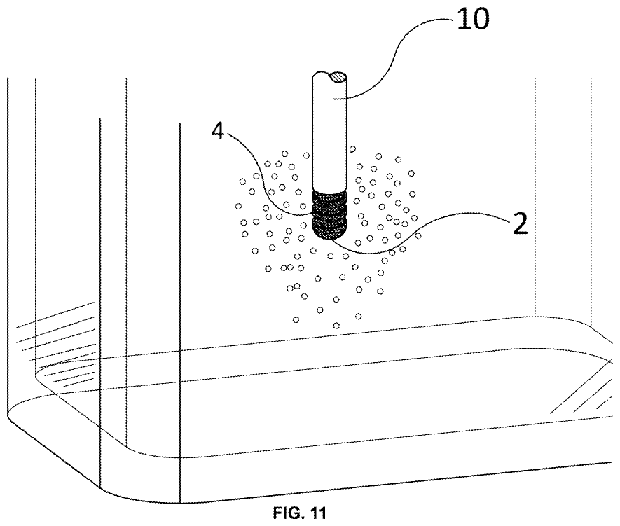

The process for the operation of ultrasonic probe 1 is as follows:
A. Use a 2-liter glass flask.
B. Pour Thinner into the flask.
C. Connect the ultrasonic probe 1 to an ultrasonic power energy generation device 20 at a power of 100.
D. The ultrasonic probe 1 is introduced
E. When introducing the ultrasonic probe 1 into the thinner, a bubble is formed that is located at the tip and goes downwards, which generates a very large active area that optimizes the ultrasound output, which causes the ultrasonic probe 1 to have a greater sound emission directed in multiple direction, as illustrated in FIG. 11.

While it has not been mentioned, one familiar with the art would realize that the device is not limited by the materials used to create each apparatus that comprises the invention. Any other material type can comprise some or all of the elements in constructing a system for removing contaminated materials from railcars for subsequent recycling of the railcars in various embodiments of the present invention.

Although the present invention has been illustrated and described herein with reference to preferred embodiments and specific examples, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. An ultrasonic probe for fragmenting or emulsifying soft tissue in a patient, the ultrasonic probe configured to be powered by an ultrasonic vibratory energy source, wherein the ultrasonic probe comprises:
a longitudinal handle having a proximal end and a distal end;
a longitudinal stem whose axis extends from the proximal end through the distal end;
an axis of the longitudinal stem aligned with a center of the longitudinal handle and extending through the proximal end to the distal end of the longitudinal stem;
a connection at the proximal end of the longitudinal handle for connecting the ultrasonic probe to an ultrasonic vibratory energy source;
a rounded tip at the distal end of the longitudinal stem; and
a plurality of grooves and rings located adjacent to the rounded tip which, together with the tip, include a roughened surface on the grooves, rings, and rounded tip that includes a plurality of peaks and valleys, wherein the grooves are (i) generally transverse to the axis of the longitudinal stem, (ii) along the axis of the longitudinal stem, and (iii) substantially circumscribing the axis of the longitudinal stem, wherein the roughened surface is configured to (i) increase a tissue contact surface area along and around sides of the rounded tip, (ii) provide an additional tissue fragmentation surface area of the tip in planes generally perpendicular to the axis of the longitudinal stem, wherein the additional tissue fragmentation surface is formed by the roughened surface located on the grooves, rings, and rounded tip, and wherein the roughened surface is configured to fragment or emulsify subcutaneous soft tissues due to the an additional tissue fragmentation surface area of the tip.

2. The ultrasonic probe in accordance with claim 1, wherein the roughened surface further comprises:
different orientations of the roughened surface according to a bisector of the angles formed by the directions of the peaks and valleys located in the grooves, rings, and tip of the ultrasonic probe.

3. The ultrasonic probe in accordance with claim 1, wherein the roughened surface is configured to extend the active or output area of the ultrasonic probe.

4. The ultrasonic probe in accordance with claim 1, wherein the plurality of peaks and valleys are configured to act as an active surface for tapping or ultrasound output.

5. The ultrasonic probe in accordance with claim 1, wherein the roughened surface is configured with an area having micro surfaces that generate a very large active area that optimizes the ultrasound output.

6. The ultrasonic probe in accordance with claim 1, wherein the roughened surface is configured with a greater surface area at different length scales.

7. The ultrasonic probe in accordance with claim 1, wherein the roughened surface is configured to increase an efficiency of the ultrasound output by means of its corresponding scales due to deviations or alterations of undulations and roughnesses in different orientations of the roughened surface.

8. The ultrasonic probe in accordance with to claim 1, wherein the roughened is configured to provide an optimization of the surface which creates a tapping zone helps to break down adipocytes.

9. The ultrasonic probe in accordance with claim 1, wherein the roughened surface is configured to allow the ultrasonic probe to consume less energy and ultrasound waves emitted from the ultrasonic probe more stable and uniform.

10. The ultrasonic probe in accordance with claim 1, wherein the roughened surface is configured to less power for operation which allows the ultrasonic probe to use less energy and generate less heat.

11. The ultrasonic probe, in accordance with claim 1, wherein the roughened surface is configured to reduce a thermal impact on the tissue of a patient by reducing a risk of burns and complications in a patient undergoing a medical procedure.

12. The ultrasonic probe in accordance with claim 1, wherein each of the plurality of grooves includes a bottom, and wherein each bottom includes which is rough and substantially parallel to the axis of the longitudinal stem.

13. The ultrasonic probe in accordance with claim 12, wherein each bottom of the plurality of grooves is U-shaped.

14. The ultrasonic probe, in accordance with claim 1, wherein the longitudinal stem is solid, is aligned with the axis of the longitudinal handle which is located along a center of the longitudinal handle.

15. The ultrasonic probe in accordance with claim 1, wherein the rounded tip is comprised of:
a blunt shape.

16. The ultrasonic probe in accordance with claim 1, wherein the plurality of peaks and valleys are randomly distributed along the tip, the plurality of grooves and rings.

17. A method of using an ultrasonic probe for fragmenting or emulsifying soft tissue in a patient, comprising;
providing an ultrasonic probe, wherein the ultrasonic probe comprises;
a longitudinal handle having a proximal end and a distal end,
a longitudinal stem whose axis extends from the proximal end through the distal end,
an axis of the longitudinal stem aligned with the center of the longitudinal handle and extending through the proximal end to the distal end of the longitudinal stem,
a connection at the proximal end of the longitudinal handle for connecting the ultrasonic probe to an ultrasonic vibratory energy source,
a rounded tip at the distal end of the longitudinal stem, and
a plurality of grooves and rings located adjacent to the rounded tip which,
together with the tip, include a roughened surface on the grooves, rings, and rounded tip that includes a plurality of peaks and valleys, wherein the grooves are (i) generally transverse to the axis of the longitudinal stem, (ii) along the axis of the longitudinal stem, and (iii) substantially circumscribing the axis of the longitudinal stem, wherein the roughened surface is configured to (i) increase a tissue contact surface area along and around sides of the rounded tip, (ii) provide an additional tissue fragmentation surface area of the tip in planes generally perpendicular to the axis of the longitudinal stem, wherein the additional tissue fragmentation surface is formed by the roughened surface located on the grooves, rings, and rounded tip, and wherein the roughened surface is configured to fragment or emulsify subcutaneous soft tissues due to the an additional tissue fragmentation surface area of the tip; and
powering by the ultrasonic vibratory energy source, the ultrasonic probe which causes the ultrasonic probe to direct energy proportional to 360° from the tip.

18. The method in accordance with claim 17, wherein the roughened surface is configured to extend the active or output area of the ultrasonic probe.

19. The method in accordance with claim 17, wherein the roughened is configured to provide an optimization of the surface which creates a tapping zone helps to break down adipocytes.

20. The method in accordance with claim 17, wherein the roughened surface is configured to reduce a thermal impact on the tissue of a patient by reducing a risk of burns and complications in a patient undergoing a medical procedure.
